(19) United States
(12) Patent Application Publication
(10) Pub. No.: US 2021/0280320 A1
(43) Pub. Date: Sep. 9, 2021

(54) Community Vulnerability Index Dashboard

(71) Applicant: Parkland Center for Clinical Innovation, Dallas, TX (US)

(72) Inventors: Akshay Arora, Irving, TX (US); Venkatraghavan Sundaram, Irving, TX (US); Lindsay Zimmerman, Dallas, TX (US); Thomas Roderick, Frisco, TX (US); Esther Olsen, Sunnyvale, CA (US); Leslie Wainwright, Chicago, IL (US); Vikas Chowdhry, Southlake, TX (US); Steve Miff, Dallas, TX (US); Aida Kreho Somun, Richardson, TX (US); Vency Varghese, Irving, TX (US)

(21) Appl. No.: 17/191,648

(22) Filed: Mar. 3, 2021

Related U.S. Application Data
(60) Provisional application No. 62/984,785, filed on Mar. 3, 2020.
(60) Provisional application No. 63/039,399, filed on Jun. 15, 2020.

(51) Int. Cl. G16H 50/80; G16H 50/30; G16H 40/20; G06Q 50/26; G06Q 30/02; G06Q 50/22; G16H 15/00; G06N 20/00

(52) U.S. Cl. CPC G16H 50/80; G16H 50/30; G16H 40/20; G06Q 50/26; G06F 3/04847; G06Q 50/22; G16H 15/00; G06Q 30/0205; G06N 20/00; G06Q 30/0201

(57) ABSTRACT

A community vulnerability index dashboard includes a data ingestion logic module configured to automatically receive real-time and non-real-time data from a variety of sources including education agencies, law enforcement, health services agencies, healthcare agencies, medical insurance agencies, housing and transportation agencies, childcare licensing agencies, and non-emergency citywide services. The dashboard includes a data processing module that extracts and process the ingested data, and a data analysis logic module that analyzes the processed data to determine values for indicators and an overall vulnerability index value based on the values of the plurality of indicators to provide insight into the lives of residents living in a community on a block group level. The dashboard includes a data presentation dashboard interface to display an interactive choropleth map of the overall vulnerability index and indicator values on a block group level for the community of interest.

### RELATED APPLICATION

This patent application claims the benefit of U.S. Provisional Patent Application No. 62/984,785 filed on Mar. 3, 2020, the entirety of which is incorporated herein by reference.

### FIELD

This disclosure relates to a dashboard analytic logic module for a computing device graphical user interface that interactively presents data insight into the quality of life and health-related vulnerabilities of a population at a block group granularity level.

### BACKGROUND

Every day, huge volumes of data are generated in various forms by disparate types of different sources. For instance, the retail giant, Wal-Mart, handles 1 million customer transactions every day, thereby adding 2.5 petabytes of information to the database. Such enormous volume of digital information is the storehouse of meaningful insights such as valuable business trends, shifting consumer behavior, onset of an epidemic, changing weather patterns and rising crime rate. When managed well, this data can provide businesses and governments an opportunity to unlock new business avenues and provide insightful solutions for better governance to improve people's lives.

Most existing data discovery platforms used to achieve insight in the collected data are either too broad and simply end up as data aggregation technical solutions, or aim too narrowly and result in static reports and dashboards for a single domain. These conventional tools fall short of providing actionable data needed by various entities to truly make significant improvements.

### BRIEF DESCRIPTION OF THE DRAWINGS

FIG. 1 is a simplified block diagram of an embodiment of the community vulnerability index dashboard hardware components according to the teachings of the present disclosure;

FIG. 2 is a simplified architectural diagram of an embodiment of the community vulnerability index dashboard according to the teachings of the present disclosure;

FIG. 3 is a simplified flow diagram of an embodiment of the community vulnerability index dashboard according to the teachings of the present disclosure;

FIG. 4 is a more detailed flow diagram of an embodiment of the community vulnerability index dashboard according to the teachings of the present disclosure;

FIGS. 5-13 and 15-17 are screen captures of an embodiment of the community vulnerability index dashboard according to the teachings of the present disclosure; and

FIG. 14 is a hierarchical diagram of the good health sub-index according to the teachings of the present disclosure.

### DETAILED DESCRIPTION

The community vulnerability index dashboard described herein is a data analytics and presentation system and method 10 that enable the users to truly understand the factors that impact the quality of life and health of various communities. The community vulnerability index dashboard system and method 10 generate highly specific, block group-level indicators from key indicator data received from a variety of publicly available data sources, and present the data analysis via a highly interactive and user-friendly geospatial graphical user interface that are adaptable for a variety of computing platforms. The dashboard system and method uses an overall community vulnerability index (CVI) and four sub-indices: 1) Household Essentials, 2) Empowered People, 3) Equitable Communities, and 4) Good Health. Each sub-index is made up of key indicators. The indices are created on the block group level and are designed to reflect both individual and neighborhood-level characteristics. The dashboard system and method provide actionable insights that enable community-based organizations, local civic leaders, and philanthropic funders to assess community needs, evaluate program effectiveness, redirect funding, apply for grants, inform key stakeholders, make and track goals, and monitor and forecast trends. The dashboard can also be incorporated into other use cases such as predictive models for health services utilization, neighborhood quality index, scenario planning, and impact analyses.

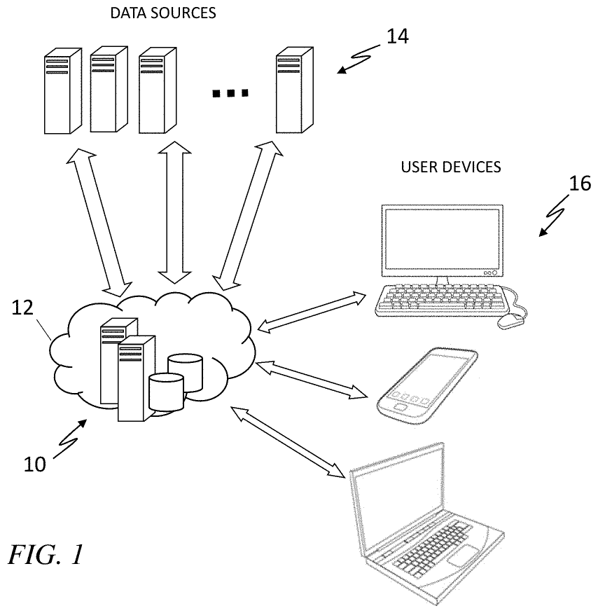

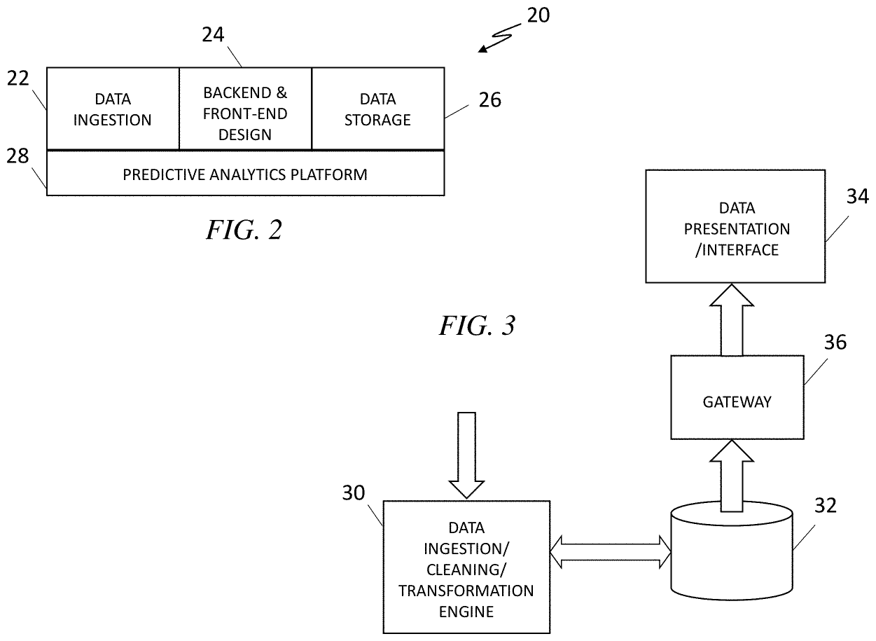

Referring to FIG. 1, The dashboard 10 is hosted, for example, on the Microsoft Azure Cloud 12. By hosting everything on a single platform in an exemplary embodiment, the dashboard 10 is a streamlined process for ingesting, cleaning, analyzing, and presenting the data. The architecture 20 includes a data ingestion layer 22, a backend and front-end design layer 24, and a storage layer 26 that sits on top of a predictive analytics platform 28 that may be hosted in the Azure infrastructure, as shown in FIG. 2.

Referring also to FIG. 3, through an automated data ingestion engine 30 using, for example, Apache NiFi, raw data is received from a variety of data sources 14 using, for example, File Transfer Protocol (FTP), Simple Object Database Access (SODA)/Application Program Interface (API), client URL (cURL), and other methods. Data is automatically pulled from the data sources 14 on a regular or periodic basis to ensure that the current data is the up to date. Real-time data, if available, may also be received and used for analysis. The data sources 14 may include (for data related to, for example, Dallas County, Tex.) Texas Education Agency, the Centers for Disease Control and Prevention, the Census, Feeding America, Department of State Health Services, Dallas Independent School District, Dallas Police Department, Texas Department of Family and Protective Services, Neighborhood Atlas, County Health Rankings & Roadmaps, Centers for Medicare and Medicaid Services, Housing and Transportation Affordability Index, Texas Health and Human Services, U.S. Department of Housing and Urban Development, Dallas County Votes, etc. The data is cleaned for quality and accuracy according to predefined scripts. Cleaned data are then moved within the Azure environment to a HIPAA-compliant database 32, such as a PostgreSQL database management system, and stored in a tabular format.

The dashboard 10 may use a data presentation/interface tool 34, for example, the Power BI dashboard tool, to pull data from the database management system 32 via a gateway 36. Power BI is a Microsoft product that can be easily integrated with the Azure platform. An overall community vulnerability index is based on five sub-indices for five main SDOH (Social Determinants of Health) categories. These sub-indices include: household essentials (indicators: food insecurity, paycheck predictability, household structure, health insurance coverage, and median income), empowered people (indicators: educational attainment, internet connectivity, literacy, and mobility), equitable communities (indicators: employment, affordable housing, neighborhood safety, neighborhood stability, clean air, and green space), good health (indicators: life expectancy, alcohol abuse, mental health, cancer, chronic diseases: coronary heart disease, diabetes, chronic obstructive pulmonary disease (COPD), kidney disease, and asthma), and access to vital services (indicators: childcare, elder care, healthcare, social services, utilities, and food). These five categories are described in more detail below. Additionally, a master community vulnerability index is available to provide users the opportunity to look at how these indicators interact across these categories. The dashboard 10 also uses a mapping application that presents data as actionable insights into the community in question covered by the data. Users may access the dashboard 10 by using a variety of user devices 16, including and not limited to, mobile phones, tablet computers, laptop computers, and desktop computers.

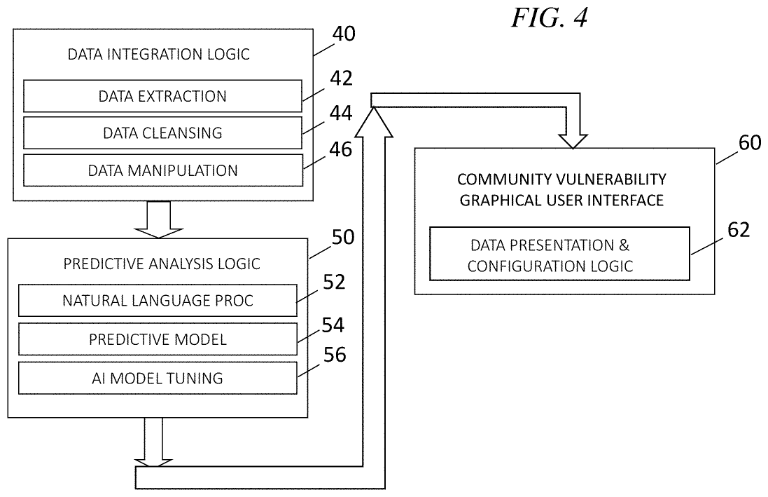

Referring to FIG. 4, a simplified flowchart of the dashboard process is shown. Through the automated pipeline, raw data are obtained electronically from various data sources 14. The data ingestion is automated and occurs periodically and/or in real-time. A data integration logic 40 includes a data extraction module/process 42, data cleansing module/process 44, and data manipulation module/process 46. The data extraction process 42 may extract data using various technologies and protocols. The data cleansing process 44 “cleans” or pre-processes the data, putting structured data in a standardized format and preparing unstructured text for natural language processing (NLP) to be performed in the predictive analysis logic module 50 described below. This logic module may also convert the data into desired formats (e.g., text date field converted to numeric for calculation purposes). The data manipulation module/process 46 may analyze the representation of a particular data feed against a meta-data dictionary and determine if a particular data feed should be re-configured or replaced by alternative data feeds.

The predictive analysis logic module/process 50 receives the data from the data integration logic module/process 40 and analyzes the data. The predictive analysis logic module/process 50 includes a natural language processing logic 52. During natural language processing, raw unstructured data, for example, physicians' notes and reports, first go through a process called tokenization. The tokenization process divides the text into basic units of information in the form of single words or short phrases by using defined separators such as punctuation marks, spaces, or capitalizations. Using the rule-based model, these basic units of information are identified in a meta-data dictionary and assessed according to predefined rules that determine meaning. Using the statistical-based learning model, the disease identification process 44 quantifies the relationship and frequency of word and phrase patterns and then processes them using statistical algorithms. Using machine learning, the statistical-based learning model develops inferences based on repeated patterns and relationships. A number of complex natural language processing functions including text pre-processing, lexical analysis, syntactic parsing, semantic analysis, handling multi-word expression, word sense disambiguation, and other functions are performed.

The predictive analysis logic module/process 50 includes a predictive model process 54 that is adapted to analyze the data and predict the risk of occurrence of particular conditions of interest according to one or more predictive models. It may be used to assess the vulnerability of a certain population with respect to certain diseases, such as, for example, alcohol abuse, mental health, cancer, coronary heart disease, diabetes, COPD, kidney disease, and asthma. The predictive model analysis takes into account of the values of risk factors or variables (weighed or unweighed) and compare them against setpoints and thresholds to determine the amount of risk certain residents in a population of a community is subject to or suffering from certain diseases. One or more predictive models may be incorporated to analyze the data and calculate risk scores associated with particular members of a certain block group in order to determine the best course of action to take with respect to those members or that block group.

Artificial Intelligence (AI) 58 may also be used to analyze the ingested data. The artificial intelligence model tuning module/process 58 utilizes adaptive self-learning capabilities using machine learning technologies. The capacity for self-reconfiguration enables the system and method to be sufficiently flexible and adaptable to detect and incorporate trends or differences in the underlying patient data or population that may affect the predictive accuracy of a given algorithm. The artificial intelligence model tuning module/process 58 may periodically retrain a selected predictive model for improved accurate outcome to allow for selection of the most accurate statistical methodology, variable count, variable selection, interaction terms, weights, and intercept for a local health system or clinic. The artificial intelligence model tuning module/process may automatically modify or improve a predictive model in three exemplary ways. First, it may adjust the predictive weights of the variables without human supervision. Second, it may adjust the threshold values of specific variables without human supervision. Third, the artificial intelligence model tuning process may, without human supervision, evaluate new variables present in the data feed but not presently used in the predictive model, which may result in improved accuracy. The artificial intelligence model tuning module/process may compare the actual observed outcome of the event to the predicted outcome then separately analyze the variables within the model that contributed to the incorrect outcome. It may then re-weigh the variables that contributed to this incorrect outcome, so that in the next reiteration those variables are less likely to contribute to a false prediction. In this manner, the artificial intelligence model tuning module/process is adapted to reconfigure or adjust the predictive model based on the specific clinical setting or population in which it is applied. Further, no manual reconfiguration or modification of the predictive model is necessary. The artificial intelligence model tuning module/process may also be useful to scale the predictive model to different populations, communities, and geographical areas in a rapid timeframe.

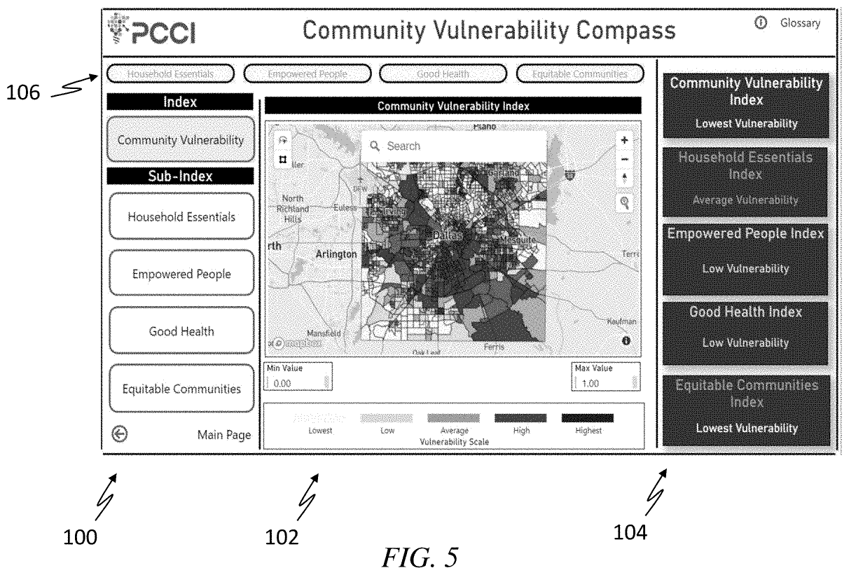

The community vulnerability index dashboard system and method 10 further includes a graphical user interface 60 that includes a data presentation and configuration logic module/process 62. The dashboard interface 60 is an interactive and user-friendly visualization tool that is designed to enable the user to understand neighborhood characteristics of a selected region or a default geopolitical region on a block group level in a holistic manner. The dashboard displays 60+ sub-indicators grouped into five categories that measure the resiliency, commitment and amenities in the neighborhoods on a block group level. FIGS. 5-12 and 14-17 show exemplary screenshots of the dashboard graphical user interface 60, which has three main components: an indicator right panel, a choropleth or heatmap, and a right panel showing average summary of the selected index for the selected area, which may be a neighborhood, street, city, county, state, multi-state regions.

FIG. 5 is an exemplary landing page for the community vulnerability dashboard. On the left sidebar 100, users can choose to display either the community vulnerability index (CVI) or any of the sub-indices (only four shown in this example). In the center is a choropleth map or heat map 102 of the community vulnerability index across a certain geographical region, such as geopolitical area of interest, colored by the CVI quintile for each of the block groups. When the user clicks on a particular category, the heat map is updated to reflect the vulnerability index for the region on a group block level. On the right sidebar 104, the average values for the community vulnerability indices are displayed for the region of interest. The dashboard home page also displays a rating of the vulnerability level based on quintiles on the block group level in the geopolitical area of interest in the five categories (as shown on right sidebar) as lowest, low, average, high, and highest with respective color coding on a block group level. A menu button bar 106 across the top of the page provides a quick way for the user to obtain insight into each category: household essentials, empowered people, good health, equitable communities.

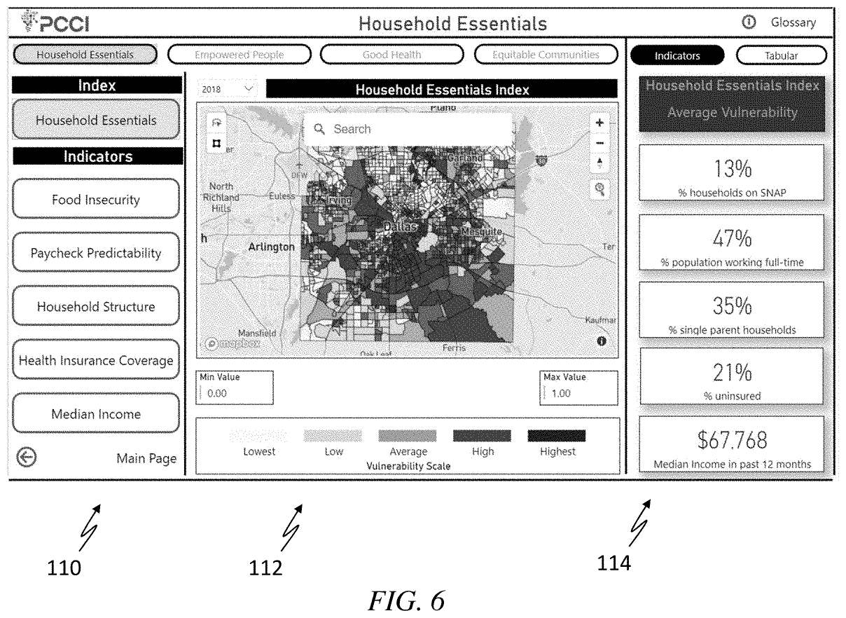

FIG. 6 is an exemplary screenshot of an exemplary page for the household essentials index. The house essentials index page captures broader measures of economic stability and access to health insurance coverage. The left sidebar 110 shows the buttons for the five indicators that make up the household essentials sub-index. The distribution of the household essentials sub-index is shown on the choropleth map 112 by block group. If users choose a different indicator on the left sidebar, the map will update to show the distribution of that indicator across the geopolitical area of interest. The right sidebar 114 provides an overall average for the sub-index and indicators for the geopolitical area of interest.

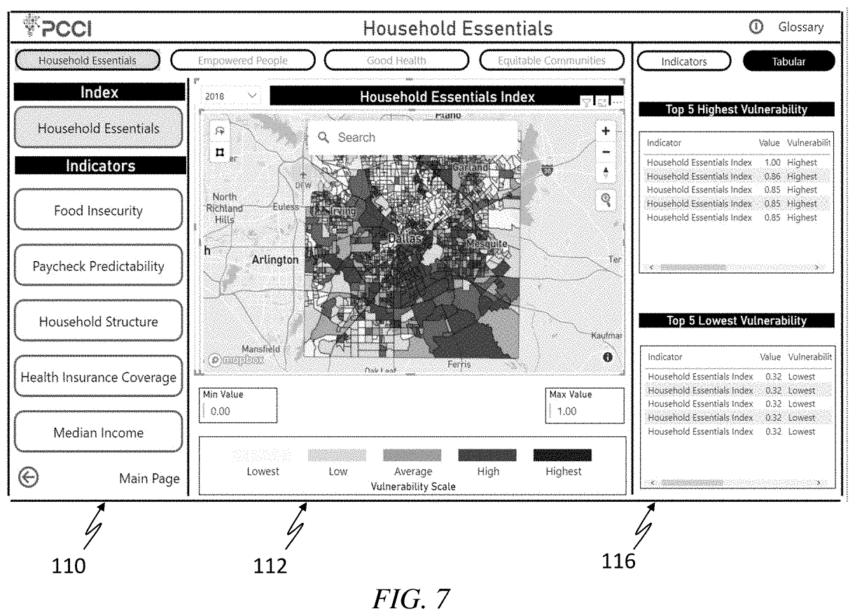

When the user clicks on the “Tabular” button on the screen shown in FIG. 7, the screen shown in FIG. 7 is displayed. The tabular panel on the right 116 displays information for the top five highest vulnerability block groups, in addition to the top five lowest vulnerability block groups. These tables contain information about the selected indicator, in addition to a cross street within the block group and corresponding zip code, county, and state to allow for better identification of the block group on the map.

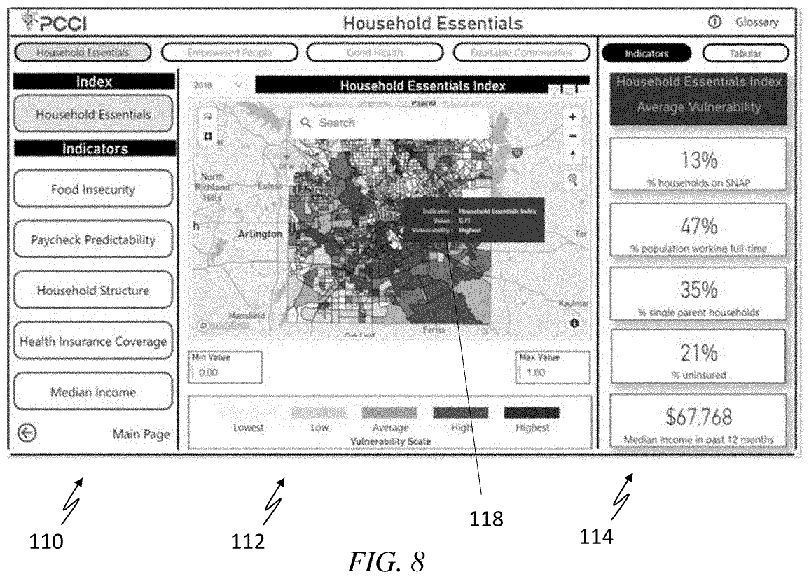

When the user hovers the cursor over a block group on the map 112, information for that specific block group 118 is displayed, as shown in FIG. 8.

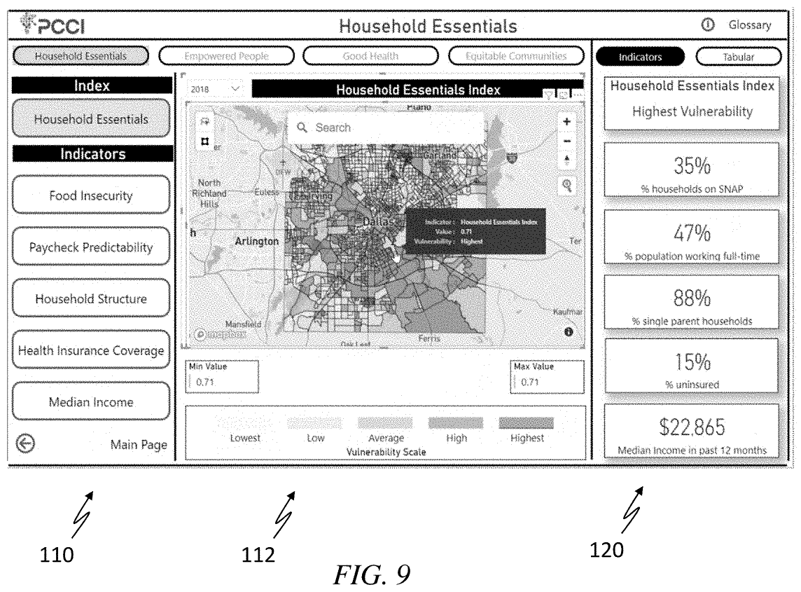

As shown in FIG. 9, after the user clicks on a specific block group, the right sidebar 120 is repopulated to show the sub-index and indicator information for the selected block group of interest.

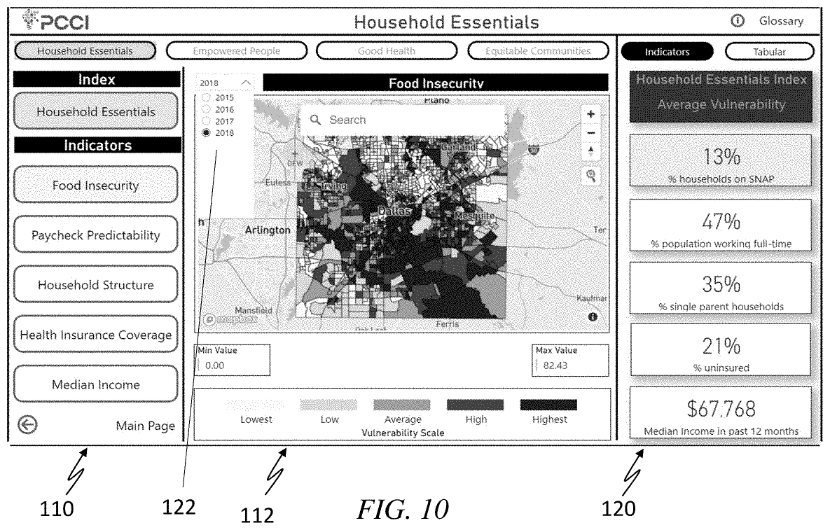

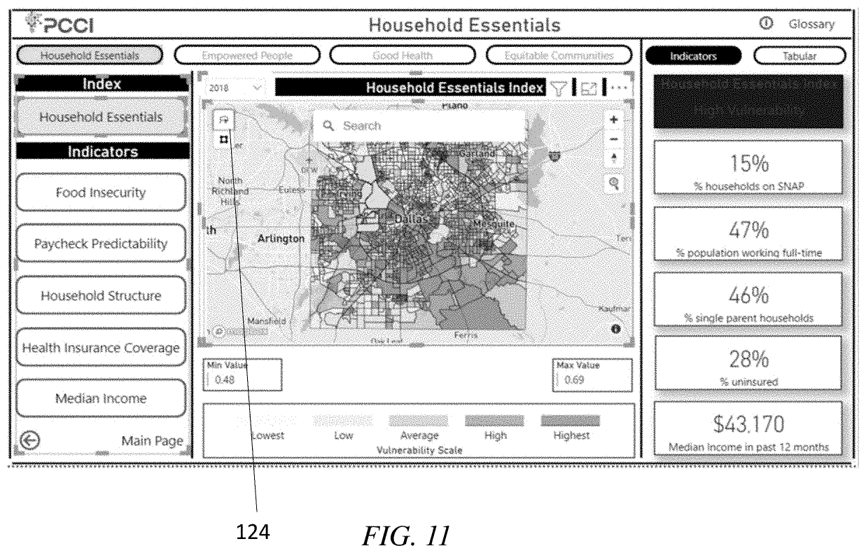

As shown in FIG. 10, users can select a particular year from a drop-down menu 122 to show vulnerability index data for a specific year of interest. As shown in FIG. 11, a lasso tool 124 is incorporated into the dashboard, which allows users to select multiple block groups. The right sidebar updates to show the average values of the sub-index and indicators for all of the block groups selected by using the lasso tool 124.

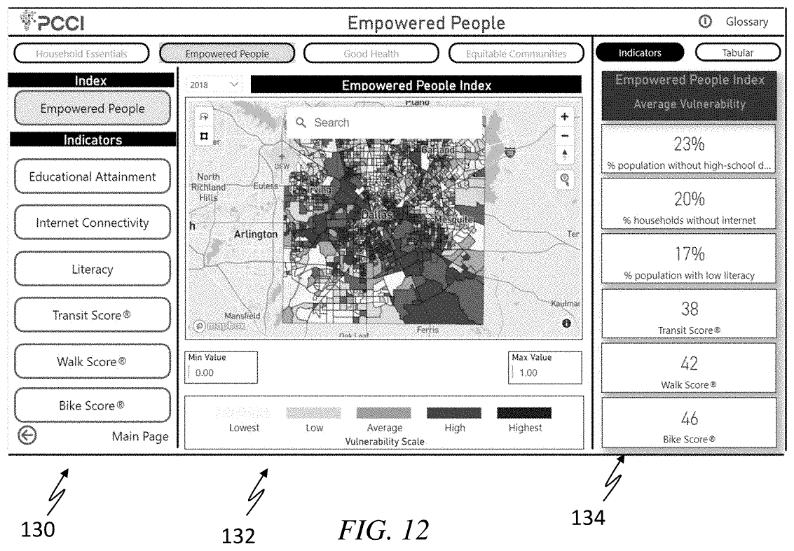

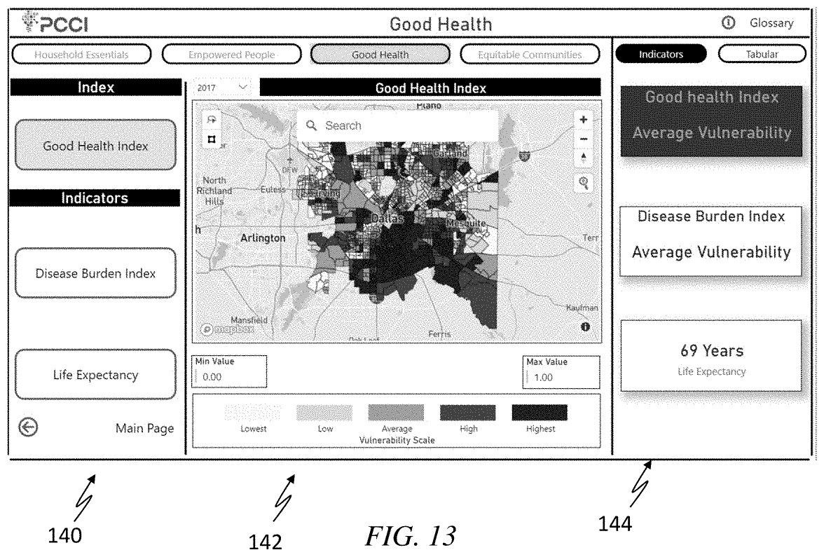

FIG. 12 shows the home page for the empowered people sub-index. The empowered people sub-index captures information related to factors that help residents live their healthiest lives possible, including education, access to internet, literacy, and mobility access. The empowered people page also features a left panel 130 with buttons for the indicators make up the empowered people index. The distribution of the empowered people sub-index is shown on the choropleth map 132 by block group. If users choose a different indicator on the left sidebar, the map will update to show the distribution of that indicator across the geopolitical area of interest. The right sidebar 134 provides an overall average for the empowered people sub-index and indicators for the geopolitical area of interest.

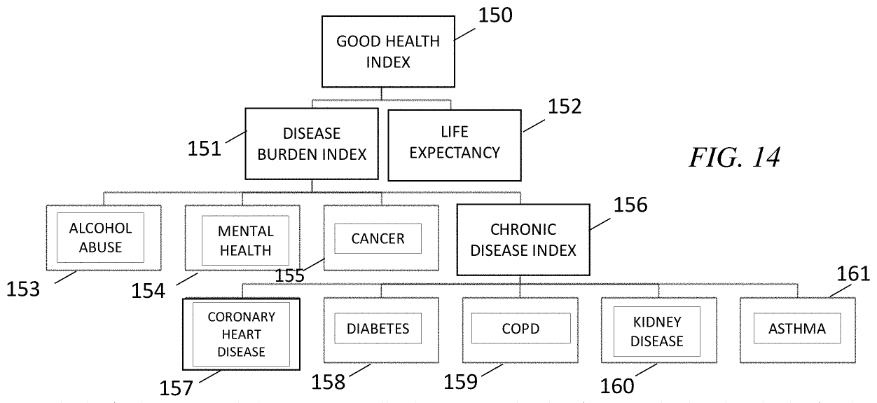

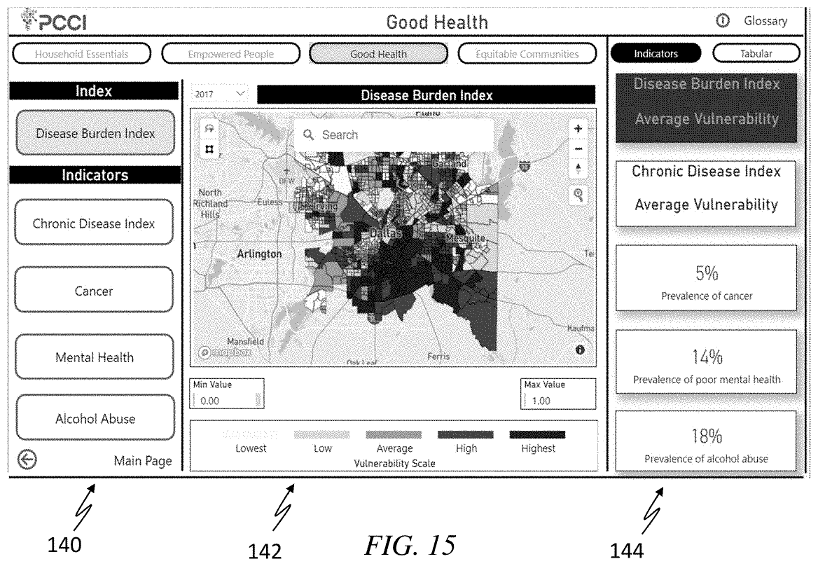

FIG. 13 shows the home page for the good health sub-index. The good health sub-index captures information related to mental health, physical health, and life expectancy. The good health page also features a left panel 140 with buttons for the indicators make up the good health index: a disease burden index and life expectancy. The distribution of the good health sub-index is shown on the choropleth map 142 by block group. If users choose a different indicator on the left sidebar, the map will update to show the distribution of that indicator across the geopolitical area of interest. The right sidebar 144 provides an overall average for the good health sub-index and indicators for the geopolitical area of interest. As shown in FIG. 14, the good health index 150 has a hierarchical structure, where a disease burden index 151 includes a number of indicators: alcohol abuse 153, mental health 154, cancer 155, and a chronic disease index 156, which in turn includes coronary heart disease 157, diabetes 158, COPD 159, kidney disease 160, and asthma 161. FIG. 15 shows an exemplary landing page for the disease burden index, which includes a chronic disease index, cancer, mental health, and alcohol abuse. The right panel displays the prevalence of cancer, poor mental health, and alcohol abuse.

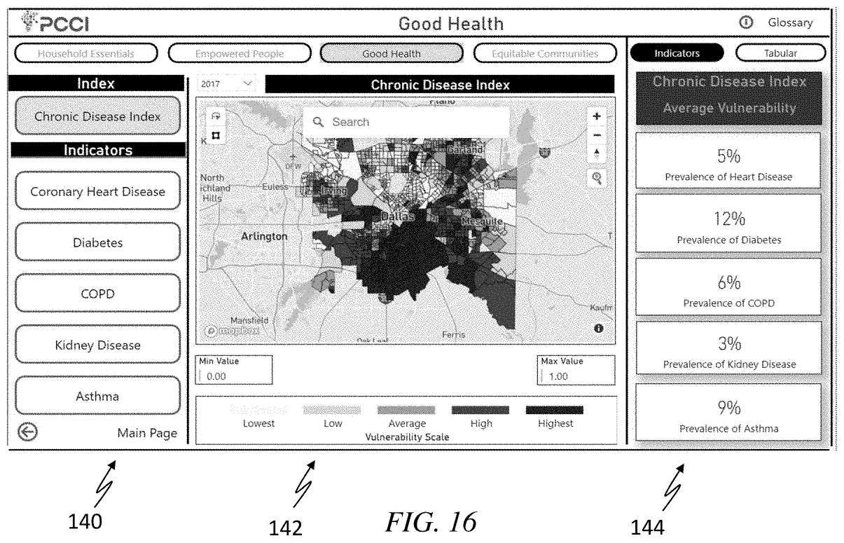

FIG. 16 shows the landing page for the chronic disease index, which shows indicators coronary heart disease, diabetes, COPD, kidney disease, and asthma. The prevalence for these chronic diseases is displayed in the right panel 144.

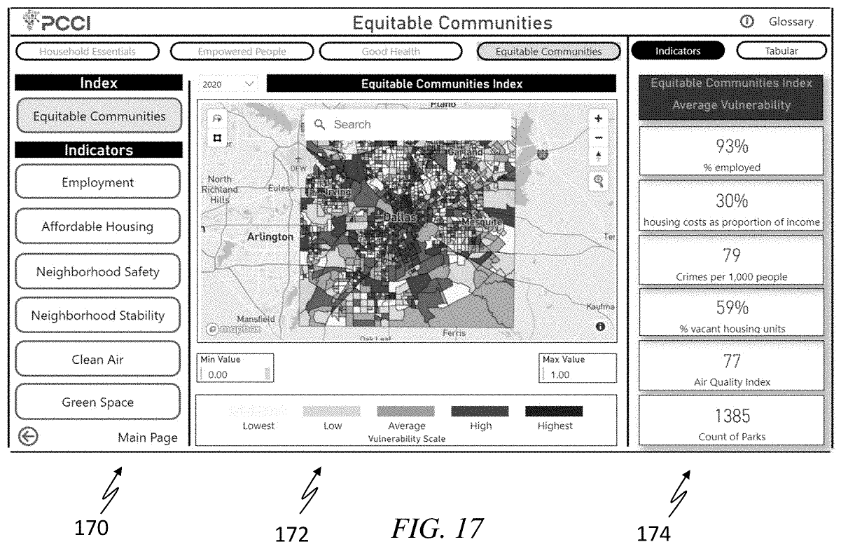

FIG. 17 is the landing page for the equitable communities sub-index. The equitable communities sub-index captures information related to the neighborhoods in which the residents live, ranging from economic, safety, and environmental factors. The left panel 170 shows the indicators that relate to equitable communities sub-index: employment, affordable housing, neighborhood safety, neighborhood stability, clean air, and green space. The distribution of the equitable communities sub-index is shown on the choropleth map 172 by block group. If users choose a different indicator on the left sidebar, the map will update to show the distribution of that indicator across the geopolitical area of interest. The right sidebar 174 provides an overall average for the equitable communities sub-index and indicators for the geopolitical area of interest.

The dashboard system and method use various key indicators that have been selected and used to track measures of resiliency, commitment, and amenities in a region. Table A lists the sub-indices, the indicators for each sub-index, and the data source for the indicators.

TABLE A

| Domain | Measure | Description | Data Source |
|---|---|---|---|
| Household | Food | % of households on | American |
| Essentials | Insecurity | SNAP in the past 12 | Community |
| | | months | Survey |
| | Paycheck | % of population | American |
| | Predictability | working full-time, | Community |
| | | year-round in the | Survey |
| | | past 12 months for | |
| | | the population 16 | |
| | | years and over | |
| | Household | % single parent | American |
| | Structure | households | Community |
| | | | Survey |
| | Health | % uninsured | American |
| | Insurance | | Community |
| | Coverage | | Survey |
| | Median | median household | American |
| | Income | income in the past 12 | Community |
| | | months (in 2018 | Survey |
| | | inflation-adjusted | |
| | | dollars) | |
| Empowered | Educational | % of the population, | American |
| People | Attainment | 25 years and over, | Community |
| | | without high school | Survey |
| | | degree | |
| | Internet | % of households | American |
| | Connectivity | without an internet | Community |
| | | subscription | Survey |
| | Literacy | % of residents with | Program for the |
| | | low literacy | International |
| | | (description of levels | Assessment |
| | | found here) | of Adult |
| | | | Competencies |
| | Walk Score ®* | score measures | Walk Score ® |
| | | walkability on a scale | |
| | | from 0-100 by | |
| | | analyzing routes to | |
| | | nearby amenities and | |
| | | pedestrian | |
| | | friendliness | |
| | Bike Score ®* | score measures | Walk Score ® |
| | | whether an area is | |
| | | good for biking by | |
| | | analyzing bike | |
| | | infrastructure, terrain, | |
| | | road connectivity, | |
| | | and number of bike | |
| | | commuters | |
| | Mobility- | score measuring | Walk Score ® |
| | Transit | transit accessibility | |
| | Score ® | on a scale from 0- | |
| | | 100 by calculating | |
| | | distance to closest | |
| | | stop on each route | |
| | | (analyzes route | |
| | | frequency and type) | |
| Equitable | Employment | % of employed | American |
| Communities | | individuals out of the | Community |
| | | civilian labor force | Survey |
| | | ages 16 years and | |
| | | older | |
| | Affordable | average monthly | H + T ® Index |
| | Housing | housing costs as a | |
| | | percentage of | |
| | | household income in | |
| | | the past 12 months | |
| | Neighborhood | all crime and violent | Dallas Crime |
| | Safety | crime rates per 1,000 | Data |
| | | residents in the past | |
| | | year | |
| | Neighborhood | % of housing units | American |
| | Stability | that are vacant | Community |
| | | | Survey |
| | Clean Air | concentration of air | BreezoMeter |
| | | pollutants based on | |
| | | local air quality | |
| | | standards and | |
| | | pollutant | |
| | | concentrations | |
| | Green Space | number of parks per | ParkServe ® |
| | | block group | |
| Good Health | Life | life expectancy at | U.S. Small- |
| | Expectancy | birth (average | area Life |
| | | number of years a | Expectancy |
| | | person can expect to | Estimates |
| | | live) | Project |
| | Alcohol | prevalence of binge | 500 Cities |
| | Abuse | drinking among | Project |
| | | adults ages 18 years | |
| | | and older | |
| | Mental | % of adults 18 years | 500 Cities |
| | Health | and older who stated | Project |
| | | that their mental | |
| | | health, which | |
| | | includes stress, | |
| | | depression, and | |
| | | problems with | |
| | | emotions, was not | |
| | | good for 14 or more | |
| | | of the past 30 days | |
| | Cancer | prevalence of cancer | 500 Cities |
| | | among adults ages 18 | Project |
| | | years and older | |
| | Coronary | prevalence of | 500 Cities |
| | Heart | coronary heart | Project |
| | Disease | disease among adults | |
| | | ages 18 years and | |
| | | older | |
| | Diabetes | prevalence of | 500 Cities |
| | | diagnosed diabetes | Project |
| | | among adults ages 18 | |
| | | years and older | |
| | Chronic | prevalence of chronic | 500 Cities |
| | Obstructive | obstructive | Project |
| | Pulmonary | pulmonary disease | |
| | Disease | among adults ages 18 | |
| | | years and older | |
| | Kidney | prevalence of chronic | 500 Cities |
| | Disease | kidney disease among | Project |
| | | adults ages 18 years | |
| | | and older | |
| | Asthma | prevalence of current | 500 Cities |
| | | asthma among adults | Project |
| | | ages 18 years and | |
| | | older | |

The features of the present invention which are believed to be novel are set forth below with particularity in the appended claims. However, modifications, variations, and changes to the exemplary embodiments described above will be apparent to those skilled in the art, and the community vulnerability index dashboard described herein thus encompasses such modifications, variations, and changes and are not limited to the specific embodiments described herein.

What is claimed is:

1. A community vulnerability index dashboard comprising:
a data ingestion logic module configured to automatically receive real-time and non-real-time data from a variety of sources selected from the group consisting of education agencies, law enforcement, health services agencies, healthcare agencies, medical insurance agencies, housing and transportation agencies, childcare licensing agencies, and non-emergency citywide services;
a data processing module configured to extract and process the ingested data;
a data analysis logic module configured to analyze the processed data to determine values for a plurality of indicators and an overall vulnerability index value based on the values of the plurality of indicators to provide insight into the lives of residents living in a community on a block group level; and
a data presentation dashboard interface to display values for the plurality of indicators and the overall vulnerability index and an interactive choropleth map of the overall vulnerability index and indicator values on a block group level for the community of interest.

2. The dashboard of claim 1, further comprising a database configured to store ingested data and provide access to the data by the data analysis logic module.

3. The dashboard of claim 1, wherein the data analysis logic module applies natural language processing techniques to process unstructured data.

4. The dashboard of claim 1, wherein the data analysis logic module is configured to analyze data indicative of food insecurity, paycheck predictability, household structure, health insurance coverage, and median income that are representative of household essentials.

5. The dashboard of claim 1, wherein the data analysis logic module is configured to analyze data indicative of educational attainment, internet connectivity, literacy, walkability, bikability, and transit availability that are representative of personal empowerment.

6. The dashboard of claim 1, wherein the data analysis logic module is configured to analyze data indicative of employment, affordable housing, neighborhood safety, neighborhood stability, clean air, and green space that are representative of equitable communities.

7. The dashboard of claim 1, wherein the data analysis logic module is configured to analyze data indicative of life expectancy, alcohol abuse, disease burden, and mental health that are representative of health.

8. The dashboard of claim 1, wherein the data analysis logic module comprises at least one predictive model having a plurality of variables and thresholds.

9. The dashboard of claim 8, wherein the data analysis logic module applies artificial intelligence methods to refine and tune the at least one predictive model.

10. A method to provide improved insight into community vulnerability by presenting data on a graphical user interface device comprising:
automatically receiving real-time and non-real-time data from a variety of sources selected from the group consisting of education agencies, law enforcement, health services agencies, healthcare agencies, medical insurance agencies, housing and transportation agencies, childcare licensing agencies, and non-emergency citywide services;
extracting and processing the ingested data;
analyzing the processed data and determining values for a plurality of indicators and an overall vulnerability index value based on the values of the plurality of indicators to provide insight into the lives of residents in a community on a block group level; and
displaying values of the plurality of indicators and the overall vulnerability index value; and
displaying an interactive choropleth map of the overall vulnerability index and indicator values on a block group level for a community of interest.

11. The method of claim 10, further comprising storing the ingested data and providing secured access to the data.

12. The method of claim 10, further comprising applying natural language processing techniques to process ingested data that are unstructured.

13. The method of claim 10, further comprising analyzing data indicative of food insecurity, paycheck predictability, household structure, health insurance coverage, and median income that are representative of household essentials.

14. The method of claim 1, further comprising analyzing data indicative of educational attainment, internet connectivity, literacy, walkability, bikability, and transit availability that are representative of personal empowerment.

15. The method of claim 1, further comprising analyzing data indicative of employment, affordable housing, neighborhood safety, neighborhood stability, clean air, and green space that are representative of equitable communities.

16. The method of claim 1, further comprising analyzing data indicative of life expectancy, alcohol abuse, disease burden, and mental health that are representative of health.

17. The method of claim 1, further comprising applying at least one predictive model having a plurality of variables and thresholds to the data.

18. The method of claim 17, further comprising employing artificial intelligence techniques to refine and tune the at least one predictive model.

19. The method of claim 10, further comprising enabling the user to selective choose a geographical region of interest for which the overall vulnerability index, the plurality of indicators, and interactive choropleth map are presented.

20. A community vulnerability index dashboard comprising:
a data ingestion logic module configured to automatically receive real-time and non-real-time data from a variety of sources selected from the group consisting of education agencies, law enforcement, health services agencies, healthcare agencies, medical insurance agencies, housing and transportation agencies, childcare licensing agencies, and non-emergency citywide services;
a data processing module configured to extract and process the ingested data;
a data analysis logic module configured to analyze the processed data to determine values for a plurality of indicators and an overall community vulnerability index value based on the values of the plurality of indicators selected from the group consisting of food insecurity, paycheck predictability, household structure, health insurance coverage, median income, educational attainment, internet connectivity, literacy, walkability, bikability, transit availability, employment, affordable housing, neighborhood safety, neighborhood stability, clean air, green space, life expectancy, alcohol abuse, disease burden, and mental health to provide insight into the lives of residents living in a community on a selectable granularity level;
a database configured to store ingested data and provide access to the data by the data analysis logic module; and
a data presentation dashboard interface to display values for the plurality of indicators and the overall vulnerability index value and an interactive choropleth map of the overall vulnerability index and indicator values on the selectable granularity level for a community of interest.
